(19) United States
(12) Patent Application Publication
(10) Pub. No.: US 2018/0008572 A1
(43) Pub. Date: Jan. 11, 2018

(54) ANTIMETASTATIC COMPOSITION COMPRISING AT LEAST ONE FLAVANOL-TYPE COMPOUND

(71) Applicants: VALORE SA, Seneffe (BE); UNIVERSITÉ CATHOLIQUE DE LOUVAN, Louvain-la-Neuve (BE)

(72) Inventors: Paul Niebes, Grez-Doiceau (BE); Henric Bronislav May, Overijse (BE); Saïd Rachidi, Hyon (BE); Julien Estager, Kontich (BE); Pierre Sonveaux, Sterrebeek (BE); Thibaut Vazeille, Tubize (BE); Paolo Ettore Porporato, Volvera (IT); Valéry Payen, Ecaussinnes (BE)

(73) Assignees: VALORE SA, Seneffe (BE); UNIVERSITÉ CATHOLIQUE DE LOUVAN, Louvain-la-Neuve (BE)

(21) Appl. No.: 15/523,983

(22) PCT Filed: Nov. 10, 2015
(86) PCT No.: PCT/EP2015/076248

§ 371 Date: May 3, 2017

(30) Foreign Application Priority Data
Nov. 10, 2014 (BE) 2014/5061

(51) Int. Cl. A61K 31/353; A61K 31/198; A61K 9/00; A61K 31/375

(52) U.S. Cl. CPC A61K 31/353; A61K 31/375; A61K 31/198; A23L 33/175; A23L 33/16; A23L 33/105; A61K 9/0053

(57) ABSTRACT

The invention relates to an anti-metastatic composition comprising at least one flavanol-type compound for the use thereof as a medicament, said at least one flavanol-type compound being present in the form of a complex formed by the creation of coordinate bonds with at least one basic amino acid and/or at least one derivative of a basic amino acid.

The present invention relates to a composition comprising at least one flavanol-type compound for use as a medicament.

Catechins are phytochemical molecules known for their anti-oxidative and chemoprotective characteristics. They are present in a large number of plant-based foods and in high concentrations in tea leaves. Epidemiological studies demonstrating the anti-carcinogenic effect of fruits and of green tea have prompted investigations into their anti-tumorigenic activity. Nevertheless, their common general structure, considerable differences in their biochemical characteristics and the consequences thereof on the bioavailability and stability of these compounds have meant that the results observed in vitro were not always representative.

The anti-metastatic activity of a composition comprising at least one flavanol-type compound is suggested in the study by Menon et al. (Cancer Letters 1999, 141: 159-165). This article indicates that melanoma cell invasion is inhibited in mice having received cancer cells by intravenous injection, when they are treated with catechin, a flavanol-type compound. This study proposes more particularly that the inhibition of melanoma cell invasion is due to an inhibitory activity of the catechin on the action of metalloproteinases, which would make it possible to block the degradation of the connective web by these enzymes and which would thus prevent the tumor cells from penetrating into a healthy organ and forming metastases therein.

Similarly, the anti-tumorigenic effect of another composition comprising at least one flavanol-type compound is described in the study by Weyant et al. (Cancer Research 2001, 61: 118-125). This study puts forward the hypothesis that the (+)-catechin would modify the adhesion of the epithelial cells to the extracellular matrix and would induce changes in the cytoskeleton of the tumor cells, the result of which would be to modulate the cancer cell migration.

Nevertheless, more recent studies demonstrate that the anti-proliferative effect of catechin is negligible compared with that of epigallocatechin-3-gallate (EGCG), which is the most abundant flavanol of green tea (Du et al. 2012, Nutrients 4: 1679-1691). There also remain many questions regarding the mode of action of these compounds in order to be able to develop the potential of these flavonoids as therapeutic agents.

While it is well known that metastatic progenitor cells, that is to say the cells responsible for metastases, require a very significant supply of energy substrates in order to ensure their survival and their proliferation in an environment which is hostile to them, the metabolic pathways controlling tumor cell dissemination during the metastatic process are still very poorly known. However, in the studies by Porporato et al. (Cell Reports 2014, 8: 54-66), it has been demonstrated that an increased production of superoxide radical by metastatic progenitor cells appears to promote their migration. More specifically, it has been demonstrated, at the mitochondrial level of the metastatic progenitor cells, that an imbalance between an excess production of superoxide radical and of reactive oxygen species (ROSs) and the antioxidant systems capable of destroying them could explain the possibility that these cancer cells disseminate to tissues remote from the starting tumor, that they colonize in order to develop metastases therein.

In order to control this mitochondrial overproduction of superoxide radical and of other reactive oxygen species (ROSs) by the metastatic progenitor cells and with the aim of inhibiting the migration thereof, it has been envisioned to use antioxidants that selectively target the mitochondria of these cells as inhibitors of mitochondrial production of superoxide. Use is currently made for example of inhibitors such as triphenylphosphonium salts of quinone molecules (mitoQs) or piperidine-derived molecules (mitoTEMPOs), the triphenylphosphonium group allowing these products to penetrate into the mitochondria. However, the toxicity of these triphenylphosphonium salts of quinone molecules or piperidine-derived molecules is largely unknown.

Furthermore, the general antioxidants with a non-targeted action for mitochondria, such as N-acetylcysteine, vitamin C and trolox, can promote tumor growth (Porporato P E and Sonveaux P, Mol. Cell. Oncol. 2015; 2: DOI: 10.4161/23723548.2014.968043) and metastatic dissemination (Piskounova et al., Nature 2015 doi: 10.1038/nature15726; Le Gal et al., Sci. Transl. Med. 2015; 7: 308re8).

Unfortunately, the anti-metastatic compositions that are currently known and comprise antioxidants make it possible to minimize metastatic progenitor cell migration only relatively insignificantly.

### SUMMARY OF THE INVENTION

The present invention relates to a composition comprising at least one flavanol-type compound, which composition is characterized in that said at least one flavanol-type compound is present in the form of a complex formed by the creation of coordinate bonds with at least one basic amino acid or at least one derivative of a basic amino acid. According to the invention, it is thus a molecular complex formed between a flavanol-type compound and at least one basic amino acid or at least one derivative of a basic amino acid by the creation of coordinate bonds.

According to a first aspect, the present invention relates to a composition comprising at least one flavanol-type compound for use in the preventive and/or curative treatment of cancer metastases, characterized in that said at least one flavanol-type compound is present in the form of a complex formed by the creation of coordinate bonds with at least one basic amino acid or at least one derivative of a basic amino acid.

In one embodiment, the composition is characterized in that said flavanol-type compound is chosen from the group consisting of (+)-catechin, (−)-catechin, (+)-epicatechin, (−)-epicatechin, (+)-epigallocatechin, (−)-epigallocatechin, (+)-epicatechin gallate, (−)-epicatechin gallate, (+)-gallocatechin, (−)-gallocatechin, (+)-gallocatechin gallate, (−)-gallocatechin gallate, and mixtures thereof. In one preferred embodiment, the composition is characterized in that said flavanol-type compound is catechin or a catechin derivative.

In one embodiment, the composition is characterized in that said complex has a molar equivalence ratio between said flavanol-type compound and said at least one basic amino acid or said at least one derivative of a basic amino acid of between 1:0.25 and 1:5, preferably between 1:1 and 1:4, preferentially between 1:1 and 1:3, preferably between 1:1 and 1:2.5, more preferentially between 1:1 and 1:2. In one preferred embodiment, the composition is characterized in that said complex has a molar equivalence ratio between said flavanol-type compound and said at least one basic amino acid or said at least one derivative of a basic amino acid of between 1:2 and 1:4, for instance 1:2.

In one embodiment, the composition is characterized in that said at least one basic amino acid is chosen from the group consisting of lysine, arginine, ornithine, citrulline, histidine, pyrolysine, tryptophan, proline, and mixtures thereof. In one preferred embodiment, said at least one basic amino acid is lysine.

In one embodiment, the composition is characterized in that it also comprises at least one acid. In one preferred embodiment, said at least one acid is chosen from the group consisting of ascorbic acid, acetic acid, citric acid, hydrochloric acid, and mixtures thereof.

In one embodiment, the composition is characterized in that said at least one flavanol-type compound present in the form of a complex is present in a molar concentration of between 0.01 μM and 5000 μM, preferably of between 0.02 μM and 2500 μM, preferentially of between 0.1 μM and 1000 μM, preferably of between 0.5 μM and 500 μM.

In one embodiment, the composition is characterized in that it also comprises one or more biocompatible excipients.

In one embodiment, the composition is characterized in that it is in liquid form or in solid form, preferably in water-soluble solid form, such as powder or a tablet or else a pessary or a suppository.

In one embodiment, the composition is characterized in that it is formulated for oral, rectal, vaginal, injectable or cutaneous use.

In one embodiment, the composition is characterized in that the content of at least one flavanol-type compound present in the form of a complex formed by the creation of coordinate bonds with at least one basic amino acid or at least one derivative of a basic amino acid is between 15% and 95% by weight relative to the total weight of said composition, preferably between 60% and 90%, advantageously from 65% to 85% relative to the total weight of said composition.

In one embodiment, the composition is characterized in that, in 0.01 molar solution at 25° C., it has a pH greater than or equal to 3, preferably of between 4 and 11, advantageously of between 4.5 and 9. In one preferred embodiment, it has a pH of between 7.2 and 7.4.

In one embodiment, the cancer is a liver, prostate, breast, uterine, testicular, bladder, kidney, lung, bronchial, bone, mouth, esophageal, stomach, pancreatic, colorectal, or else brain cancer. In one embodiment, the cancer is a hepatocellular cancer. In one embodiment, the cancer is a leukemia, a myeloma, a lymphoma or a melanoma.

According to another aspect, the invention relates to an anti-metastatic composition comprising at least one flavanol-type compound for use as a medicament, characterized in that said at least one flavanol-type compound is present in the form of a complex formed by the creation of coordinate bonds with at least one basic amino acid or at least one derivative of a basic amino acid. In one embodiment, the composition is characterized in that said at least one flavanol-type compound present in the form of a complex is present in a molar concentration of between 0.01 μM and 5000 μM, preferably of between 0.02 μM and 2500 μM, preferentially of between 0.1 μM and 1000 μM, preferably of between 0.5 μM and 500 μM. In one embodiment, the composition is characterized in that said flavanol-type compound is chosen from the group consisting of (+)-catechin, (−)-catechin, (+)-epicatechin, (−)-epicatechin, (+)-epigallocatechin, (−)-epigallocatechin, (+)-epicatechin gallate, (−)-epicatechin gallate, (+)-gallocatechin, (−)-gallocatechin, (+)-gallocatechin gallate, (−)-gallocatechin gallate, and mixtures thereof. In one embodiment, the composition is characterized in that said at least one basic amino acid is chosen from the group consisting of lysine, arginine, ornithine, citrulline, histidine, pyrolysine, proline and tryptophan, and mixtures thereof. In one embodiment, the composition is characterized in that said complex has a molar equivalence ratio between said flavanol-type compound and said at least one basic amino acid or said at least one derivative of a basic amino acid of between 1:0.25 and 1:5, preferably between 1:1 and 1:4, preferentially between 1:1 and 1:3, preferably between 1:1 and 1:2.5, more preferentially between 1:1 and 1:2. In one embodiment, the composition is characterized in that it also comprises one or more biocompatible excipients. In one embodiment, the composition is characterized in that it can also comprise an acid. In one embodiment, the composition is characterized in that said acid is chosen from the group consisting of ascorbic acid, acetic acid, citric acid, hydrochloric acid, and mixtures thereof. In one embodiment, the composition is characterized in that it is in liquid form or in solid form, preferably in water-soluble solid form, such as powder or a tablet or else a pessary or a suppository. In one embodiment, the invention comprises the composition as described above, for oral, rectal, vaginal, injectable or cutaneous use. In one embodiment, the composition is characterized in that the content of at least one flavanol-type compound present in the form of a complex formed by the creation of coordinate bonds with at least one basic amino acid or at least one derivative of a basic amino acid is between 15% and 95% by weight relative to the total weight of said composition, preferably between 60% and 90%, advantageously from 65% to 85% relative to the total weight of said composition. In one embodiment, the composition is characterized in that, in 0.01 molar solution at 25° C., it has a pH greater than or equal to 3, preferably of between 4 and 11, advantageously of between 4.5 and 9. In one embodiment, the invention relates to the composition as described above, for the preventive and/or curative treatment of liver, prostate, breast, uterine, testicular, bladder, kidney, lung, bronchial, bone, mouth, esophageal, stomach, pancreatic, colorectal or else brain cancers. In one embodiment, the invention relates to the composition as described above, for the preventive and/or curative treatment of cancer, preferably of hepatocellular cancer. In one embodiment, the invention relates to the composition as described above, for the preventive and/or curative treatment of leukemias. In one embodiment, the invention relates to the composition as described above, for the preventive and/or curative treatment of myelomas. In one embodiment, the invention relates to the composition as described above, for the preventive and/or curative treatment of lymphomas. In one embodiment, the invention relates to the composition as described above, for the preventive and/or curative treatment of melanomas. The invention also relates to the use of a composition as described above, for the production of an anti-metastatic medicament.

The independent and dependent claims define the particular and preferred features of the invention. The features of the dependent claims can be combined with the features of the independent claims or with other dependent claims as appropriate. The attached claims are also explicitly included by way of reference in the present description.

### DETAILED DESCRIPTION OF THE INVENTION

Before describing the present invention in greater detail, it should be understood that this invention is not limited to the particular processes, compositions, uses, components, products or combinations described, these compositions, uses, processes, components, products or combinations possibly of course varying. It is also understood that the terminology used herein could not be considered to be restricted, given that the field of application of the present invention will have as limit only the attached claims.

As used herein, the singular forms “a”, “an” and “the” include the singular and plural references except if the context clearly indicates the contrary.

The terms “comprising”, “comprises” and “composed of” as used herein are synonyms of “including”, “includes”, “containing” or “contains”, and are inclusive or open and do not exclude the additional members, elements or process steps not mentioned. It should not be forgotten that the terms “comprising”, “comprises” and “composed of” as used herein comprise the terms “constituted of”, “consists in” and “consists of”, and also the terms “essentially consisting of”, “essentially consisting in” and “essentially consists of”.

The mention of numerical value ranges by their extreme points includes all the numbers and fractions integrated into the respective ranges, and also the extreme points recited.

The term “about” or “approximately” as used herein, when applied to a measurable value such as a parameter, an amount, a time period, and others that are similar, means that there is a degree of variation of ±20% or less, preferably of ±10% or less, more preferentially of ±5% or less, and even more preferentially of ±1% or less relative to the value specified, insofar as these variations are appropriate for implementing the present invention. It is understood that the value to which the description “about” or “approximately” refers is itself also specifically and preferably described.

Although the terms “one or more” or “at least one”, such as one or more or at least one member(s) of a group of members, is clear as such, by means of a demonstration by example, the term includes, inter alia, a reference to any one of said members, or to at least any two of said members, for instance ≧3, ≧4, ≧5, ≧6 or ≧7, etc., of said members, and up to all said members.

All the references cited in the present description are incorporated herein by way of reference in their entirety. In particular, the teachings of all the references expressly mentioned herein are incorporated by way of reference.

Unless they are defined otherwise, all the terms used in the description of the invention, including the technical and scientific terms, have the meaning commonly understood by any person skilled in the art of which the invention is part. As additional information, definitions of the terms are included for better understanding of the teaching of the present invention.

In the following paragraphs, various aspects of the invention are defined in greater detail. Each aspect thus defined can be combined with any one or other aspects unless clearly indicated otherwise. In particular, any feature indicated as being preferred or advantageous can be combined with one or other features indicated as being preferred or advantageous.

Any reference in this description to “one embodiment” means that a particular function, structure or feature described relative to the embodiment is included in at least one embodiment of the present invention. Thus, the occurrences of the phrase “in one embodiment” in various places in this description do not all necessarily refer to the same embodiment, but may do so. Moreover, the particular functions, structures or features can be combined in any appropriate manner, as would appear obvious to those skilled in the art on reading this description, in one or more embodiments. Moreover, although some embodiments described herein comprise some but not other features included in other embodiments, the combinations of features of the various embodiments are in the field of application of the invention, and form various embodiments, as will be understood by those skilled in the art. For example, in the attached claims, any one of the claimed embodiments can be used in any combination.

In the following detailed description of the invention, reference is made to the appended figures which are an integral part of the invention, and in which are represented, solely by way of illustration, the specific embodiments in which the invention can be carried out. It is understood that other embodiments can be used and structural or logical modifications can be introduced without departing from the field of application of the present invention. The following detailed description should not therefore be considered to be limiting, and the field of application of the present invention is defined by the attached claims.

As used herein, the expression “composition comprising at least one flavanol-type compound”, for the purposes of the present invention, refers to a composition comprising one or more flavanol-type compounds. This comprises but is not limited to a composition comprising mainly or consisting exclusively of at least one flavanol compound.

For the purposes of the present invention, the term “anti-metastatic composition” is intended to mean a composition which prevents or at least minimizes the formation of metastases, that is to say which prevents or at least minimizes the dissemination (migration) and/or invasion and/or growth and/or proliferation of a tumor cell (metastatic progenitor cell) remote from the site initially affected and in a tissue or an organ reached by the blood or lymphatic circulation. Similarly, as used herein, the expression “a method for anti-metastatic treatment” is intended to mean, for the purposes of the present invention, a treatment which prevents or at least minimizes the formation of metastases, that is to say which prevents or at least minimizes the dissemination (migration) and/or invasion and/or growth and/or proliferation of a tumor cell (metastatic progenitor cell) remote from the site initially affected and in an organ reached by the blood or lymphatic circulation.

The term “patient” is intended to mean any animal which is requiring a treatment according to the invention. This comprises both animals, more particularly mammalian animals, and human beings.

The term “metastatic progenitor cells” is intended to mean, for the purposes of the present invention, cells which are responsible for metastases.

The term “a derivative of a basic amino acid” is intended to mean, for the purposes of the present invention, any chemical derivation of a basic amino acid, for instance an addition of a —CH₃ or —C₂H₅ group or else of an amine group.

Flavanols, 3-flavanols, flavan-3-ols or else catechins are a subfamily of flavonoids which are secondary metabolites of plants all sharing one and the same basic structure formed by two aromatic rings connected by three carbons: C₆-C₃—C₆.

The term “a catechin derivative” is intended to mean, for the purposes of the present invention, a catechin of which one or more of its OH functions is derivatized, for example by alkoxylation or by acylation, for instance tetramethoxycatechin, pentaacetoxycatechin, and cyclic derivatives.

The flavonols are present in the compositions according to the invention in the form of a complex formed by the creation of coordinate bonds with at least one basic amino acid or at least one derivative of a basic amino acid. Reference is made to a complex formed by the creation of coordinate bonds since the weak bases and acids, such as (+)-catechin, are characterized by the fact that, in solution, more than 99% of their concentration is in non-ionic form, which means very few ions in solution and thus no salt formation.

In the context of the present invention, it has been observed, entirely surprisingly, that a flavanol-type compound, in the form of a complex formed by the creation of coordinate bonds with at least one basic amino acid or at least one derivative of a basic amino acid, makes it possible to provide a composition which can be used as an anti-metastatic composition, that is to say which can be used as a medicament for inhibiting the migration of metastatic progenitor cells at the start of a primary tumor.

It has moreover been shown that this composition is not toxic, does not block mitochondrial respiration of healthy cells and acts as an agent which modulates the production of superoxide and of reactive oxygen species produced by the metastatic progenitor cells, which constitutes a definite advantage for use as a medicament in particular for human beings.

Firstly, it has been observed, surprisingly, that a flavanol-type compound in the form of a complex according to the invention makes it possible to regulate (that is to say to modulate) an excessive production, by metastatic progenitor cells, of superoxide and of reactive oxygen species without however, inhibiting or completely eliminating their presence which is naturally required in healthy cells, in particular in order to ensure correct communication between cells and for correct functioning of said cells.

Secondly, it has been shown, for the first time, that a flavanol-type compound in the form of a complex according to the invention is able to induce a significant reduction in the number of metastatic progenitor cells migrating from a primary tumor to a healthy organ.

The term “migration” or “dissemination” is intended to mean, for the purposes of the present invention, the movement of metastatic progenitor cells from an original site (primary tumor) to other parts of the body (using the lymphatic system and/or the blood system).

It is in the context of the present invention that, entirely surprisingly and for the first time, a flavanol-type compound in the form of a complex according to the invention has been identified as being an inhibitor which makes it possible to reduce by at least 5% to 10%, preferably by at least 20% to 40%, preferentially by at least 50% to 75%, the number of metastatic progenitor cells migrating from a primary tumor to a healthy organ, this flavanol-type compound in the form of a complex according to the invention modulating the production of superoxide and of reactive oxygen species (ROSs) produced by these cancer cells.

Without wishing to be in any way limited by theory, it is estimated that a part of the effect could be attributed to the effect on the production of superoxide and of reactive oxygen species (ROSs) by the mitochondria of metastatic progenitor cells. It should be noted that the prior art is completely silent with regard to this mode of action of modulation by a flavanol-type compound in the form of a complex on production of superoxide and of reactive oxygen species by metastatic progenitor cells. This mode of action is all the more surprising since, even though flavanol-type compounds are known to be antioxidant compounds that are particularly powerful even at low concentration, they are also recognized as having a very low membrane penetration capacity. Consequently, it was certainly not expected that such flavanol-type compounds, even in the form of a complex as provided by the present invention, would be able to penetrate through the mitochondrial membranes of the metastatic progenitor cells in order to modulate therein an excessive production of superoxide and of reactive oxygen species (ROSs) and to thus have a significant impact on reducing the number of migrating metastatic progenitor cells, which has only been demonstrated in the context of the present invention.

In all cases and in particular in the case of metastases, it is preferable for the scavenger and the modulator of the excessive production of free radicals by metastatic progenitor cells to arrive at the correct site, at the correct time and at the correct concentration and to act sufficiently quickly while at the same time being non-toxic to the cell machinery. In the context of the present invention, it has been determined, against all expectations, that a flavanol-type compound, in the form of a complex formed by the creation of coordinate bonds with at least one basic amino acid or at least one derivative of a basic amino acid, meets all these criteria perfectly.

More particularly, it has been demonstrated, in the context of the present invention, that a flavanol-type compound in the form of a complex according to the invention makes it possible to act directly and rapidly at the level of the primary tumor by appropriately modulating the presence of mitochondrial superoxide radical and of ROSs produced by the metastatic progenitor cells by thus significantly minimizing the migration of said cells. Assuming that a flavanol-type compound in the form of a complex according to the invention is capable of penetrating through the mitochondrial membrane, this would allow this compound to capture an appropriate proportion of the mitochondrial superoxide radical and of the ROSs produced by the mitochondria of the metastatic progenitor cells. According to the observations made by the inventors, the capturing and modulation of the mitochondrial superoxide radical and of the ROSs take place appropriately in order to respect the natural physiological equilibrium of the presence of mitochondrial superoxide radical and of ROSs in healthy cells.

This is particularly advantageous since the problem of dissemination (migration) of these metastatic progenitor cells is attacked at source by targeting the circulating tumor cells and by consequently inhibiting their migration to healthy organs and tissues and the formation and metastases, this being without any toxicity being observed and without affecting the healthy cells. Consequently, as long as the action of the flavanol-type compound present in the form of a complex according to the invention occurs at least partly by targeting metastatic progenitor cells in the blood or lymphatic system, the risk of said cells actually reaching a healthy organ or tissue is significantly minimized.

Independently of any theory, it has been established that the compositions according to the invention have a pronounced anti-metastatic effect in a recognized model of metastasis. In one embodiment, it has been established that a composition comprising a complex of (+)-catechin present in the form of a complex formed by the creation of coordinate bonds with at least one basic amino acid or at least one derivative of a basic amino acid has a particularly high anti-metastatic effect. In one particularly envisioned embodiment, the (+)-catechin is present in the form of a complex with a basic amino acid such as lysine or proline, or a derivative of lysine or of proline. Indeed, basic amino acids play an essential role in the composition of collagen and are essential for the formation of collagen crosslinks.

In one embodiment, the composition comprises a complex of a catechin with at least one basic amino acid, in which the flavanol is chosen from the group consisting of (+)-catechin, (−)-catechin, (+)-epicatechin, (−)-epicatechin, (+)-epigallocatechin, (−)-epigallocatechin, (+)-gallocatechin, (−)-gallocatechin, (+)-gallocatechin gallate, (−)-gallocatechin gallate, and mixtures thereof. It has been established that the anti-metastatic effect of a composition according to the invention, that is to say comprising at least one flavanol-type compound in the form of a complex according to the invention, is much higher compared with epigallocatechin gallate, which is the flavanol considered in the prior art to be the most powerful. In one embodiment, the flavanol is (+)-catechin.

In one embodiment, the composition also comprises other active compounds, for instance one or more other flavanols or other therapeutic compounds.

Finally, before the present invention, there was nothing to suggest that a flavanol-type compound in the form of a complex according to the invention could act as an anti-metastatic agent. As indicated above but without wishing to be limited by this theory, it is highly likely that this complex acts at least partly as a modulator of radicals of this type and on the ROSs from metastatic progenitor cells, in such a way that the migration of said cells is inhibited.

These observations are all the more surprising since a flavanol-type compound is recognized as having low bioavailability and since, consequently, an action by this compound not only in the organism but also subsequent to its assimilation via the digestive tract, was definitely not guaranteed. It was in fact not possible to foresee the effect of modulation by the flavanol-type compound, even present in the form of a complex as envisioned by the present invention, on the superoxide radical and on the ROSs from the metastatic progenitor cells since flavanol-type compounds are recognized as having particularly low bioavailability.

In this respect, in the context of the present invention, it has also been observed, surprisingly, that the complex formed between the flavanol-type compound and at least one basic amino acid or at least one derivative of a basic amino acid makes it possible to improve, against all expectations, the bioavailability of the composition according to the invention even though the solubility of the composition is also increased via the formation of such a complex. Indeed, as long as the solubility in an aqueous medium is increased, it is expected that the capacity of the complex to cross lipid tissues will be low; however, in the context of the present invention, it has been determined, against all expectations, that the bioavailability is nevertheless increased, also entirely surprisingly, despite the fact that the solubility is increased.

The invention thus relates to the compositions comprising at least one flavanol-type compound present in the form of a complex with a basic amino acid and/or at least one derivative of a basic amino acid and to the use thereof. Preferably, said at least one flavanol-type compound present in the form of a complex is present in a molar concentration of between 0.01 μM and 5000 μM, preferably of between 0.02 μM and 2500 μM, preferentially between 0.1 μM and 1000 μM, preferably of between 0.5 μM and 500 μM. Such a molar concentration of the flavanol-type compound present in the form of a complex has been determined as being appropriate for the latter to act as an agent which modulates (regulates) the presence of mitochondrial superoxide radical and of ROSs produced in excess by metastatic progenitor cells. Preferably, these molar concentrations correspond to a concentration of flavanol-type compound present in the form of a complex of between 0.0002 mg/ml and 400 mg/ml according to the molecular weight of the flavanol-type compound in question.

Advantageously, said flavanol-type compound is chosen from the group consisting of (+)-catechin, (−)-catechin, (+)-epicatechin, (−)-epicatechin, (+)-epigallocatechin, (−)-epigallocatechin, (+)-epicatechin gallate, (−)-epicatechin gallate, (+)-epigallocatechin gallate, (−)-epigallocatechin gallate, (+)-gallocatechin, (−)-gallocatechin, (+)-gallocatechin gallate, (−)-gallocatechin gallate, and mixtures thereof. Preferably, the flavanol is chosen from the group consisting of (+)-catechin, (−)-catechin, (+)-epicatechin, (−)-epicatechin, (+)-epigallocatechin, (−)-epigallocatechin, (+)-epicatechin gallate, (−)-epicatechin gallate, (+)-gallocatechin, (−)-gallocatechin, (+)-gallocatechin gallate, (−)-gallocatechin gallate, and mixtures thereof. Preferably, the flavanol-type compound is (+)-catechin.

Advantageously, said at least one basic amino acid is chosen from the group consisting of lysine, arginine, ornithine, citrulline, histidine, pyrolysine, tryptophan, proline and mixtures thereof. Preferably, said at least one basic amino acid is chosen from the group consisting of lysine, arginine and proline. More particularly, in one embodiment, the basic amino acid is lysine.

Preferably, said complex has a molar equivalence ratio between said flavanol-type compound and said at least one basic amino acid or said at least one derivative of a basic amino acid of between 1:0.25 and 1:5, preferably between 1:1 and 1:4, preferentially between 1:1 and 1:3, preferably between 1:1 and 1:2.5, more preferentially between 1:1 and 1:2. Such molar equivalence ratios between said flavanol-type compound and said at least one basic amino acid have been determined, in the context of the present invention, as making it possible to provide a complex that is suitable for inhibiting the migration of the metastatic progenitor cells.

Preferably, said molar equivalence ratio is greater than or equal to 1:1, in particular greater than 1:1.

Advantageously, said molar equivalence ratio is less than or equal to 1:3.

Advantageously, said molar equivalence ratio is equal to 1:2 or between 1:2 and 1:4, more preferentially between 1:2 and 1:3.

In particular, said molar equivalence ratio is less than 1:3, more particularly less than or equal to 1:2.5.

Preferentially, said molar equivalence ratio is less than 1:2.

Alternatively, the molar equivalence ratio is between 1:1.5 and 1:2.5, preferably between 1:1.5 and 1:2.

Advantageously said molar equivalence ratio is greater than or equal to 1:1 and less than or equal to 1:2.50.

Very advantageously, said molar equivalence ratio is greater than or equal to 1:1 and less than or equal to 1:1.5.

Preferably, said molar equivalence ratio is greater than or equal to 1:1.5 and less than or equal to 1:2.

Preferably, said molar equivalence ratio is greater than or equal to 1:1.5 and less than or equal to 1:2.5.

Preferentially, said composition according to the invention also comprises one or more biocompatible excipients. For example, said composition may comprise one or more lubricants, preservatives, stabilizers, sweeteners, adhesives, adjuvants, dyes, disaggregating agents, bulking agents and permeation activators. The excipients are well known by those skilled in the art.

Advantageously, said composition according to the invention may also comprise an acid. The presence of this acid makes it possible to adjust the pH of the composition (as formulated or as formed just before administration) to low values, substantially lower than the natural acidity of the complex. In one embodiment, the pH of the composition is about from 4.0 to 6.0, thereby allowing stabilization of the complex. In one embodiment, the pH of the composition is higher, up to a physiological pH value of about 7.2 to 7.4. In one embodiment, the pH of the composition is adjusted, thus making it possible to stabilize the composition in its galenic form, for example in solution at a physiological pH value of about 7.2 to 7.4. The pH of the composition can also depend on the use of the composition as a medicament, more particularly it can depend on the route of administration envisioned. In one embodiment, the preparation of the compound according to the invention in an injectable form, which can optionally be to be prepared extemporaneously before use, is for example envisioned.

The acid envisioned for reducing the value of the pH in this context can be an inorganic acid, which can be chosen from the group consisting of hydrochloric acid, sulfuric acid or phosphoric acid, or can be an organic acid such as aliphatic, cycloaliphatic, aromatic or heterocyclic acids, which are carboxylic or sulfonic, for example acetic acid, propionic acid, succinic acid, glycolic acid, gluconic acid, lactic acid, malic acid, tartaric acid, citric acid, ascorbic acid, gluronic acid, maleic acid, fumaric acid, pyruvic acid, aspartic acid, glutamic acid, mandelic acid, methanesulfonic acid, ethanesulfonic acid, pantothenic acid, beta-hydroxypropionic acid, beta-hydroxybutyric acid, malonic acid and galacturonic acid.

Preferably, said acid is chosen from the group consisting of ascorbic acid, acetic acid, citric acid, hydrochloric acid, and mixtures thereof. Preferably said acid is hydrochloric acid.

In one embodiment, the composition may comprise, in order to improve the solubility and the stability of the compounds, one or more pharmaceutically acceptable acid or base addition salts or solvates thereof. It may be advantageous to use α- or γ-cyclodextrins or derivatives thereof, in particular cyclodextrins substituted with a hydroxyalkyl, for example 2-hydroxypropylcyclodextrin or else sulfobutylcyclodextrin. In one embodiment, the composition may also comprise solvents such as alcohols, which can improve the solubility and/or the stability of the compounds.

Advantageously, the composition according to the invention is in liquid form or in solid form. In one embodiment, the composition is in water-soluble solid form, such as powder or a tablet or else a pessary or a suppository.

In one embodiment, the composition is envisioned as a combined preparation of the constituents (flavanol, amino acid, and optionally acid and excipients) for simultaneous use or for combination directly before administration. More particularly, this type of formulation is envisioned for oral administration. For example, the composition can be formulated in tablet form. In these embodiments, said complex forms post-administration.

Advantageously, said composition according to the invention is used orally, rectally, vaginally, by injection or cutaneously.

Preferably, the composition according to the invention is characterized in that the content of at least one flavanol-type compound, present in the form of a complex formed by the creation of coordinate bonds with at least one basic amino acid or at least one derivative of a basic amino acid, is between 15% and 95% by weight relative to the total weight of said composition, preferably between 60% and 90%, advantageously from 65% to 85% relative to the total weight of said composition.

Advantageously, the composition according to the invention is characterized in that, in 0.01 molar solution at 25° C., it has a pH greater than or equal to 3, preferably of between 4 and 11, advantageously of between 4.5 and 9.

According to another aspect, the present invention relates to uses of the composition according to the invention for the preventive and/or curative treatment of cancer diseases.

Preferably, the composition according to the invention is used for the preventive and/or curative treatment of liver, prostate, breast, uterine, testicular, bladder, kidney, lung, bronchial, bone, mouth, esophageal, stomach, pancreatic, colorectal or else brain cancers.

Preferably, the composition according to the invention is used for the preventive and/or curative treatment of cancer, preferably of hepatocellular cancer.

Preferably, the composition according to the invention is used for the preventive and/or curative treatment of leukemias.

Preferably, the composition according to the invention is used for the preventive and/or curative treatment of myelomas.

Preferably, the composition according to the invention is used for the preventive and/or curative treatment of lymphomas.

Preferably, the composition according to the invention is used for the preventive and/or curative treatment of melanomas.

The present invention also relates to the use of a composition according to the invention for the prevention or treatment of cancer metastases. The invention thus relates, inter alia, to the production of an anti-metastatic medicament.

In one embodiment, the invention envisions the treatment of patients who are suffering from or who risk suffering from cancer metastases. More particularly, in one embodiment, these are patients who have been diagnosed with a tumor.

In one embodiment, the tumor is a breast, liver, prostate, uterine, testicular, bladder, kidney, lung, bronchial, bone, mouth, esophageal, stomach, pancreatic, colorectal or else brain cancer tumor.

In one embodiment, the invention envisions treatment of circulating tumor cells (OTCs), that is to say the tumor cells present in the blood and in the lymph. Preferentially, among the OTCs, the composition targets the metastatic progenitor cells, that is to say the cells undergoing migration.

In one embodiment, the invention envisions the treatment of patients who have been diagnosed with a tumor with a high risk of metastasis.

In one embodiment, the tumor is a hypoxic tumor. The hypoxic nature can be demonstrated histochemically, by detection of hypoxia markers, for instance CAIX or HIF-1 alpha, or by using a hypoxia indicator such as EF5 or a hypoxia tracer such as [18F]-fluoromisonidazole.

In one embodiment, the tumor is a tumor expressing a metastasis marker. Several metastasis markers have been described in the prior art, for example Ned9, Pyk2, CPE-delta, CAIX, and metalloproteases 2 and/or 9 and also CAIX or HIF-1 alpha.

In one embodiment, the invention envisions the treatment of patients who have been diagnosed with one or more metastases.

In one embodiment, the treatment envisioned according to the invention is a combination with one or more anticancer treatments. These treatments comprise, for example, but are not limited to, surgery, radiotherapy, chemotherapy, targeted treatments, hormone therapy and immunotherapy. The composition can, namely, be administered before, during and/or after another anticancer treatment.

In one embodiment, the treatment envisioned according to the invention is a combination with a compound with an anti-proliferative action.

In one embodiment, the treatment envisioned according to the invention is a combination with a medicament or an established treatment against cancer with an antioxidant action and/or which has an effect on ROS production.

In one embodiment, the treatment envisioned comprises the administration of a composition according to the invention orally, rectally, vaginally, by injection or cutaneously. In one preferred embodiment, the administration is oral.

The compositions of the invention can be administered preoperatively, perioperatively or postoperatively. The dosage is generally determined on the basis of the size, weight and/or body surface area of the patient according to dosage regimes. In one embodiment, the composition can be administered once a day, twice a day, three times a day or four times a day. In one embodiment, the administration of a dosage of 50 mg to 2 g/day/patient can be envisioned. More particularly, a dosage of about 250 mg to 750 mg/day, for example 500 mg/day, can be envisioned. It is particularly advantageous to formulate the pharmaceutical compositions envisioned in unit dosage regime form in order to facilitate administration and uniformity of the dosage regime. The unit dosage regime form in the present document refers to physically distinct units that can serve as unit doses, each unit containing a predetermined amount of active ingredient.

Other features, details and advantages of the invention will emerge from the examples given hereinafter, in a non-limiting manner and with reference to the appended figures (graphs).

### BRIEF DESCRIPTION OF THE FIGURES

FIGS. 1A, 1B and 1C are graphs which reproduce respectively the percentage of SiHa-F3 tumor cells (F3) surviving (FIG. 1A), the percentage of cells having migrated (FIG. 1B) and the DCFDA fluorescence/protein content ratio (FIG. 10) for a treatment at variable concentrations of (+)-catechin (+C).

FIGS. 2A, 2B and 2C are graphs which reproduce respectively the percentage of SiHa-F3 tumor cells (F3) surviving (FIG. 2A), the percentage of cells having migrated (FIG. 2B) and the DCFDA fluorescence/protein content ratio (FIG. 20) for a treatment at variable concentrations of the (+)-catechin/lysine complex according to a 1:1 molar equivalence ratio.

FIGS. 3A and 3B are graphs which reproduce respectively the percentage of SiHa-F3 tumor cells (F3) surviving (FIG. 3A) and the percentage of cells having migrated (FIG. 3B) for a treatment of variable concentrations of the (+)-catechin/lysine complex according to a 1:2 molar equivalence ratio.

FIGS. 4A, 4B, 4C and 4D are graphs which reproduce the cell survival (measured by the crystal violet [CV] technique and expressed as % of non-treated SiHa-F3 supermetastatic cells) of tumor cells treated with epigallocatechin gallate (EGCG), (+)-catechin, (+)-catechin/lysineHCl 1:1 or (+)-catechin/lysineHCl (1:2) (concentrations of 100 nM, 1 μM, 10 μM, 100 μM, 500 μM or 1 mM).

FIGS. 5A, 5B, 5C and 5D are graphs which reproduce the cell survival (measured by the MTT technique and expressed as % of non-treated SiHa-F3) of tumor cells treated with epigallocatechin gallate (EGCG), (+)-catechin, (+)-catechin/lysineHCl 1:1 or (+)-catechin/lysineHCl (1:2) (concentrations of 100 nM, 1 μM, 10 μM, 100 μM, 500 μM or 1 mM).

FIGS. 6A, 6B, 6C and 6D are graphs which reproduce the DCFDA fluorescence/protein content ratio for a treatment of the SiHa-F3 tumor cells with epigallocatechin gallate (EGCG), (+)-catechin, (+)-catechin/lysineHCl 1:1 or (+)-catechin/lysineHCl (1:2) (concentrations of 100 nM, 1 μM, 10 μM, 100 μM, 500 μM or 1 mM).

FIGS. 7A, 7B, 7C and 7D are graphs which reproduce the cell migration (as % of non-treated SiHa-F3) of tumor cells treated with epigallocatechin gallate (EGCG), (+)-catechin, (+)-catechin/lysineHCl 1:1 or (+)-catechin/lysineHCl (1:2) (concentrations of 100 nM, 1 μM, 10 μM, 100 μM, 500 μM or 1 mM).

FIGS. 8A and 8B are photographs (A) and a graph (B) demonstrating the effect of (+)-catechin/lysineHCl 1:2 on melanoma tumor cells treated with rotenone (Rot 20 nM+C/L 1:2 10 μM) compared to treatment with rotenone all on its own (Rot 20 nM) on the formation of lung metastases in an experimental model. A group of animals was treated with DMSO alone and serves as a negative control.

FIGS. 9A and 9B are photographs (A) and a graph (B) showing the effect of (+)-catechin/lysineHCl 1:2 (Rot 20 nM+C/L 1:2 10 μM) and epigallocatechin-gallate/lysineHCl 1:2 (Rot 20 nM+EGCG/L 1:2 10 μM) on melanoma tumor cells during the formation of lung metastases in an experimental model. A group of animals was treated with rotenone alone (Rot 20 nM) and serves as a positive control. A group of animals was treated with DMSO alone and serves as a negative control.

FIGS. 10A and 10B are photographs (A) and a graph (B) showing the effect of catechin/lysineHCl 1:2 (Rot 10 nM+C/L 1:2 10 μM) or of epigallocatechin gallate (Rot 10 nM+EGCG 10 μM) on the formation of lung metastases in an experimental model.

### EXAMPLES

Tests of cell migration and of survival of the treated cells were carried out with a model of super-invasive tumor cells obtained from a human cervical adenocarcinoma cell line, SiHa. The selection of the super-invasive SiHa-F3 cells (F3 on the graphs) was carried out after three consecutive invasions in vitro in permeable supports of Transwell® type coated with Matrigel® (Porporato P E et al., Cell Rep 2014, 8:754-766). Such a selection of particularly aggressive metastatic progenitor cells makes it possible to validate all the better the most active anti-metastatic products.

The actual migration of the tumor cells was measured in a Boyden chamber with 50 000 cells per well and 0.15% of fetal bovine serum (FBS) used as chemoattractant. The pore diameters of the porous membrane separating the two compartments of the Boyden chamber were fixed at 8 μm. After 16 hours of migration, the cells were first fixed with methanol for 3 min and then stained with crystal violet. The culture medium used was DMEM 4.5 g/l of glucose, GlutaMAX®, 1% of a solution of penicillin/streptomycin and 10% of fetal bovine serum.

The cell survival, in order to exclude any toxic effect of the products tested, was estimated either on the basis of the quantification of the cells stained with crystal violet, or by measuring the mitochondrial succinate dehydrogenase (SDH) activity on the reduction of the MTT tetrazolium salt (suspension of formazan obtained in DMSO and measurement of absorbance at 570 nm).

Moreover, an evaluation of the impact of the products tested on the level of reactive oxygen species (ROSs) observed was carried out according to the DCFDA technique after 60 minutes of pretreatment of the SiHa-F3 metastatic progenitor cells with the products tested.

In the figures, F3 signifies: control (non-treated cells); F3+C×M signifies: cells treated with (+)-catechin at a molar concentration of x; F3 C/L 1:1×M signifies: cells treated with the (+)-catechin/lysine complex according to a 1:1 molar equivalence ratio at a molar concentration of x; F3 C/L 1:2×M signifies: cells treated with the (+)-catechin/lysine complex according to a 1:2 molar equivalence ratio at a molar concentration of x.

In order to discuss the results obtained, the molar concentration of 1 μM was considered even though other values are reproduced on the graphs. This concentration is the most relevant in terms of the production of a medicament for which it is systematically sought to minimize the concentration of active compound. It remains no less the case that the other molar concentrations are just as suitable for the production of such a medicament, all the more so since the flavanol-type compounds have low toxicity and these concentrations are thus an integral part of the present invention.

### Example 1: Treatment at Variable Concentrations of (+)-Catechin (+C)

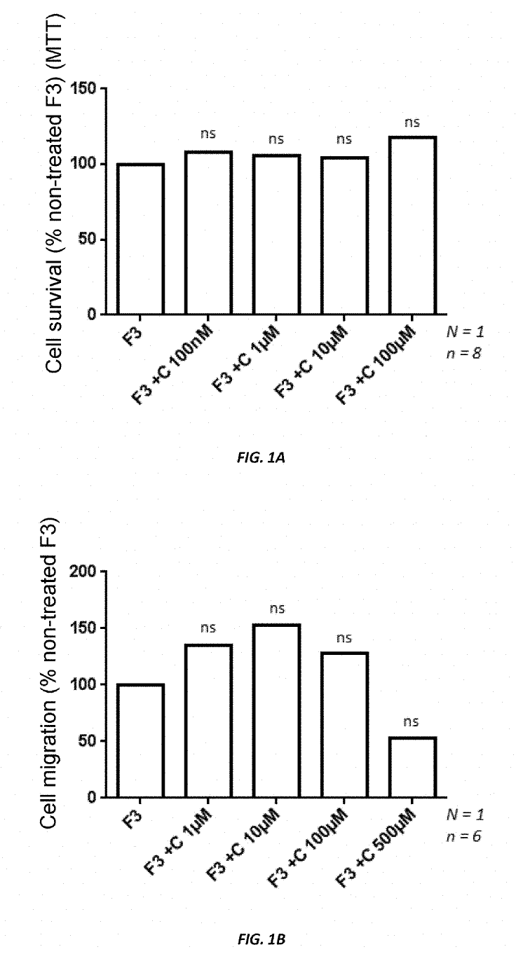

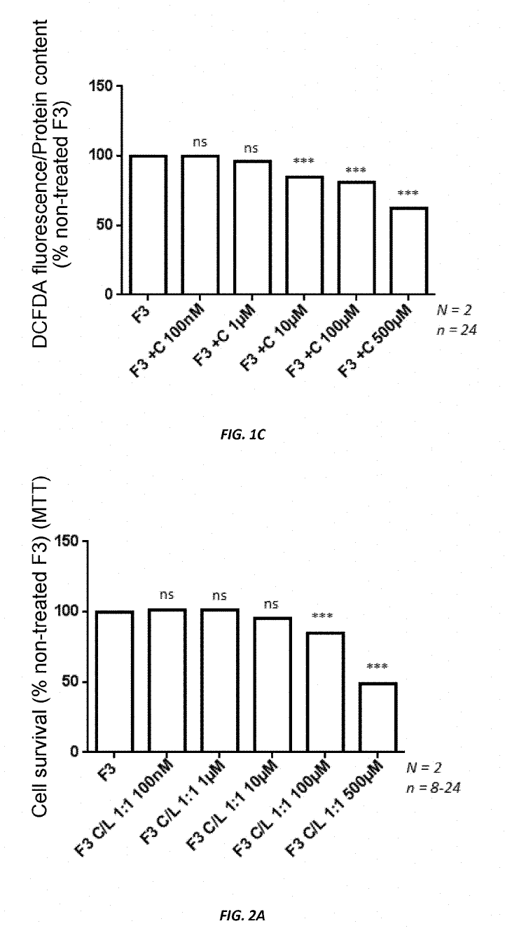

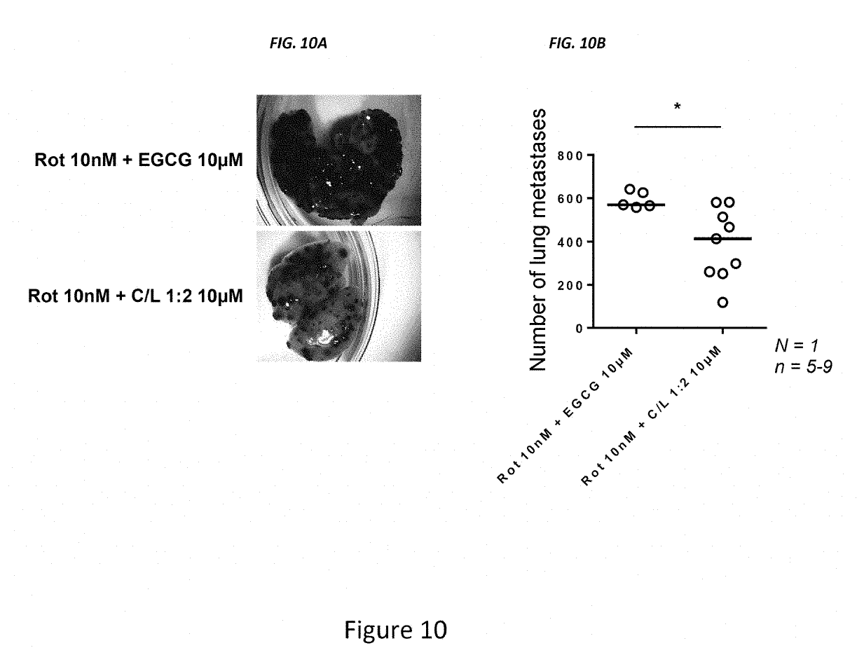

As can be noted in FIG. 1A, a molar concentration of 1 μM of (+)-catechin (+C) does not affect the survival of the tumor cells, there by indicating that, at this concentration, the (+)-catechin is not toxic. FIG. 1B makes it possible to note that a molar concentration of 1 μM of (+)-catechin does not however make it possible to significantly decrease the number of tumor cells migrating from one compartment to the other of the Boyden cell. In the same sense, FIG. 10 shows that the (+)-catechin at this same concentration of 1 μM has no impact on the level of reactive oxygen species (ROSs) observed.

### Example 2: Treatment at Variable Concentrations of the (+)-Catechin/Lysine Complex According to a 1:1 Molar Equivalence Ratio

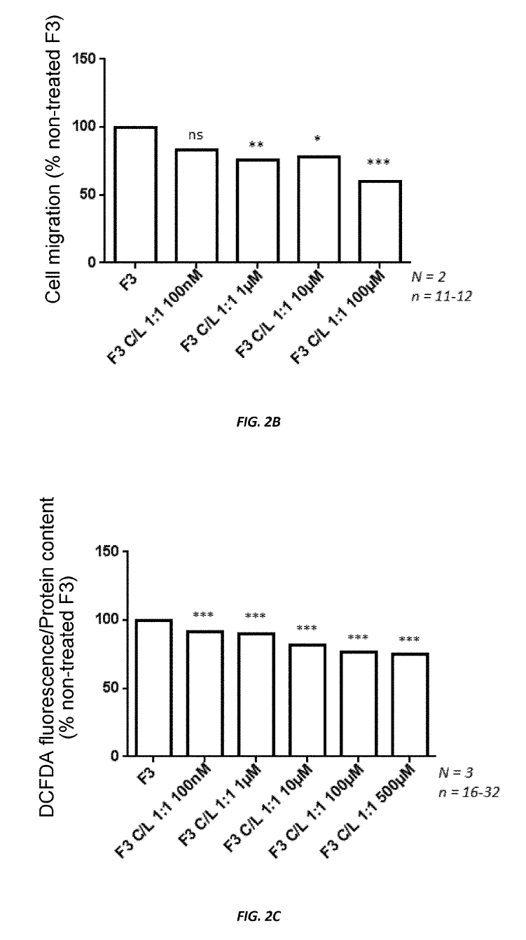

As can be noted in FIG. 2A, a molar concentration of 1 μM of the (+)-catechin/lysine complex according to a 1:1 molar equivalence ratio (C/L 1:1) does not affect the survival of the tumor cells, thereby indicating that, at this concentration, the C/L 1:1 complex is not toxic. Moreover, FIG. 2B makes it possible to note that a molar concentration of 1 μM of C/L 1:1 complex makes it possible to significantly decrease by up to 30% the number of tumor cells migrating from one compartment to the other of the Boyden cell.

It was determined that this decrease in the percentage of migrating tumor cells is linked to a reduction of about and of at least 10% in the level of reactive oxygen species (ROSs) produced by the cells, this being under the effect of the C/L 1:1 complex.

### Example 3: Treatment at Variable Concentrations of the (+)-Catechin/Lysine Complex According to a 1:2 Molar Equivalence Ratio

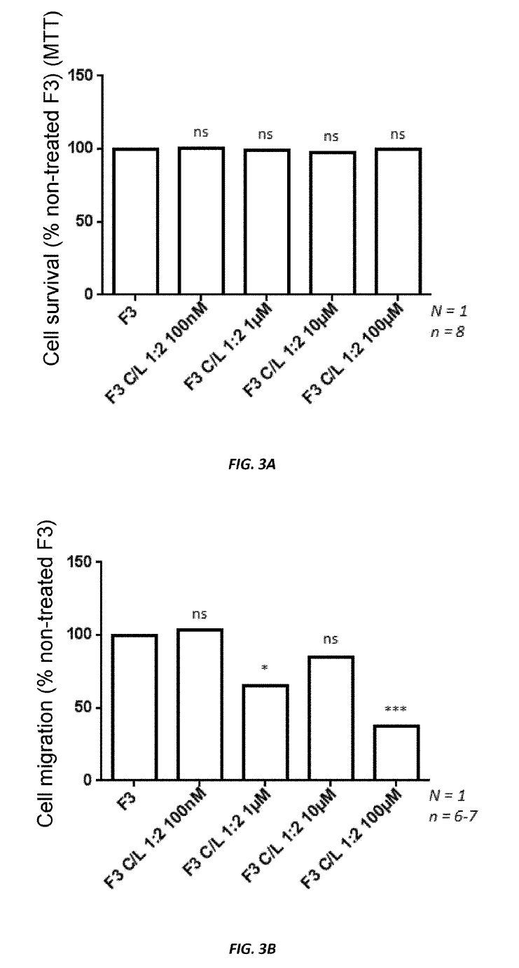

As can be noted in FIG. 3A, a molar concentration of 1 μM of the (+)-catechin/lysine complex according to a 1:2 molar equivalence ratio (C/L 1:2) does not affect the survival of the tumor cells, thereby indicating that, at this concentration, the C/L 1:2 complex is not toxic. Moreover, FIG. 3B makes it possible to note that a molar concentration of 1 μM of C/L 1:2 complex makes it possible to significantly decrease by up to 40% the number of tumor cells migrating from one compartment to the other of the Boyden cell.

It is clearly understood that the present invention is in no way limited to the embodiments described above and that many modifications can be introduced therein without departing from the context of the appended claims.

### Example 4: Cytotoxicity of the (+)-Catechin/lysineHCl Derivatives Compared to Epigallocatechin Gallate on the Tumor Cells

The epigallocatechin gallate comes from Sigma Chemical Co, St Louis, the (+)-catechin was prepared pharmaceutically pure from Bloc Gambir and its complexes were obtained by combining therewith lysine hydrochloride in molar proportion.

The SiHa-F3 cells were initially generated as described in the article recently published in Cell Reports: Porporato P E et al., Cell Rep 2014, 8:754-766. The cells were maintained in culture and manipulated in DMEM, 4.5 g/I glucose, glutaMAX (Gibco), supplemented with 10% of fetal bovine serum.

Measurement of cell viability: 20 000 SiHa-F3 cells were seeded and treated with the indicated compounds overnight (16 h). The cells were then fixed for 3 min with methanol, stained with 0.23% crystal violet, and resuspended in DMSO in order to measure the absorbance at 595 nm (Victor X4 spectrophotometer, Perkin Elmer); or alternatively incubated for 3 h at 37° C. in a saturating solution of MTT in HBSS containing 10 mM of HEPES, and resuspended in DMSO in order to measure the absorbance at 630 nm (Victor X4 spectrophotometer, Perkin Elmer).

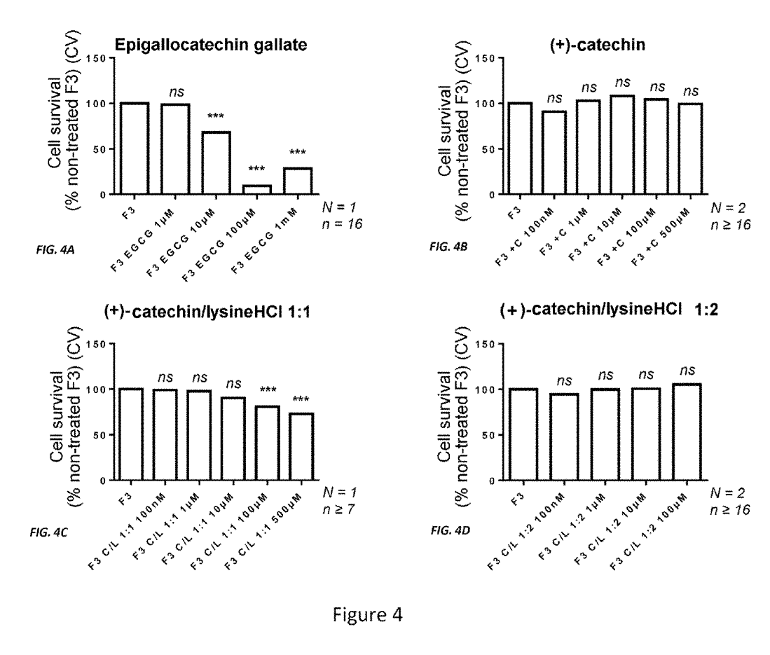

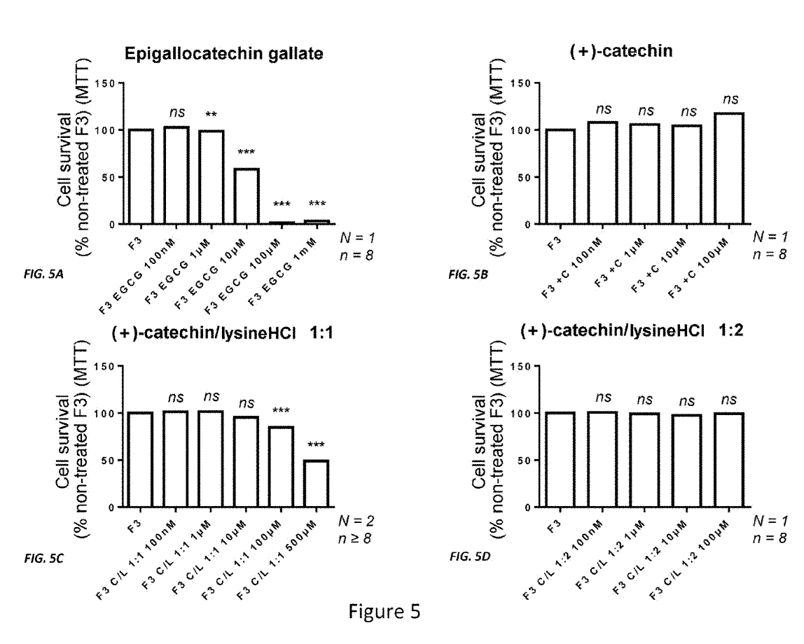

The SiHa-F3 cells were treated overnight (16 h) with epigallocatechin gallate (EGCG), (+)-catechin, (+)-catechin/lysineHCl 1:1 or (+)-catechin/lysineHCl (1:2) (concentrations of 100 nM, 1 μM, 10 μM, 100 μM, 500 μM or 1 mM) after which the cell survival was measured by staining with crystal violet (FIG. 4) or with MTT (FIG. 5).

As demonstrated in FIGS. 4 and 5, the catechin derivatives are less cytotoxic than epigallocatechin gallate.

### Example 5: Antioxidant Activity of the Compounds

The epigallocatechin gallate, the (+)-catechin and its complexes combined with lysine hydrochloride and D,L-lysine and also the SiHa-F3 cells were obtained as described above.

Measurement of total cell ROSs: 20 000 SiHa-F3 cells were seeded before being treated for 1 h with the compounds indicated. The cells were then incubated for 30 min at 37° C. in a 1 μM solution of H2-DCFDA (Invitrogen) in HBSS containing 10 mM of HEPES, before measurement of the absorbance at 535 nm (Victor X4 spectrophotometer, Perkin Elmer).

The SiHa-F3 cells were treated for 2 h with epigallocatechin gallate (EGCG), (+)-catechin, (+)-catechin/lysineHCl 1:1 or (+)-catechin/lysineHCl (1:2) (concentrations of 100 nM, 1 μM, 10 μM, 100 μM, 500 μM or 1 mM), after which the cell production of ROSs was measured by fluorescence with DCFDA.

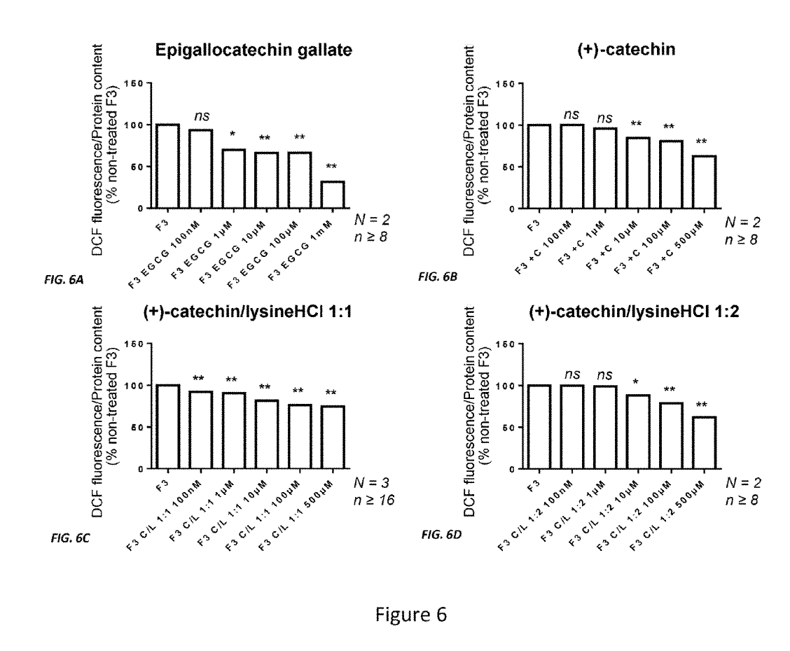

As can be noted in FIG. 6, all the catechin derivatives and the epigallocatechin gallate have a similar antioxidant activity on the tumor cells.

### Example 6: The (+)-Catechin/lysineHCl Complexes Inhibit the Tumor Cell Migration

The epigallocatechin gallate, the (+)-catechin and its complexes combined with lysine hydrochloride and D,L-lysine and also the SiHa-F3 cells were obtained as described above.

Cell migration measurements: A 48-well reusable Boyden chamber (Neuroprobe) was used according to the manufacturer's instructions. Briefly, medium containing 0.15% of fetal bovine serum served as chemoattractant in the lower compartment, a polycarbonate membrane pierced with pores 8 μm in diameter separated the two compartments, and 50 000 SiHa-F3 cells in suspension in the presence of the indicated compounds were introduced into the upper compartment. The migration of the cells was quantified after an overnight period (16 h). The cells, having migrated, were fixed for 3 min in methanol, stained with 0.23% crystal violet for 30 min, and counted on photographs taken at a magnification of ×2.5 using an inverted-phase microscope (Axiovert and Mrc camera, Zeiss).

The SiHa-F3 cells were treated during a migration performed in a Boyden chamber with epigallocatechin gallate (EGCG), (+)-catechin, (+)-catechin/lysineHCl 1:1 or (+)-catechin/lysineHCl (1:2) (concentrations of 100 nM, 1 μM, 10 μM, 100 μM, 500 μM or 1 mM), after which the number of cells having migrated toward 0.15% of serum were counted. Results were expressed as % of the control.

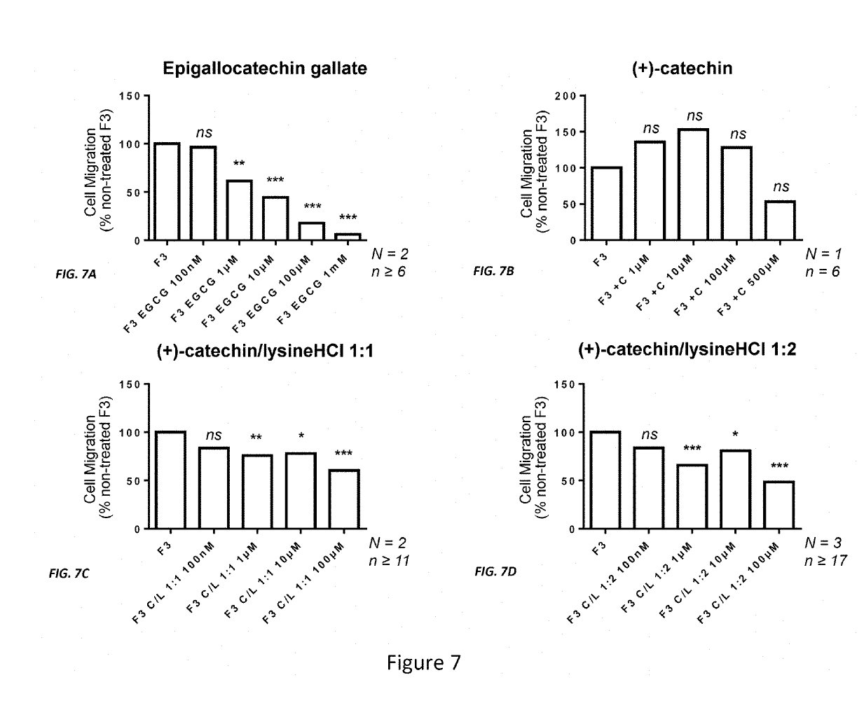

As can be noted in FIG. 7, the epigallocatechin gallate and the (+)-catechin/lysineHCl complexes, but not the (+)-catechin, inhibit the migration of the cancer cells. Only the (+)-catechin/lysineHCl complex compounds inhibit the migration of the tumor cells independently of cell cytotoxicity that would have affected the count.

### Example 7: H-Catechin/lysineHCl 1:2 Inhibits the Formation of Metastases

The B16F10 tumor cells come from the ATCC collection. The male 6- or 7-week-old C57BL/6 mice were acquired from the Janvier laboratories, and the protocols were applied with the agreement of the UCL ethics committee and while adhering to Belgian laws relating to animal care. Model of experimental metastases: the B16F10 cells were pretreated with DMSO (0.5%, rotenone carrier, negative control) or rotenone (respiratory chain complex I inhibitor and mitochondrial superoxide inducer as described in Porporato P E et al., Cell Rep 2014, 8:754-766, positive control), alone or in combination with the indicated compounds for 6 h. The cells were then detached, counted and resuspended in a proportion of 10×10⁶ cells/ml in HBSS without calcium or magnesium. 10⁶ cells (i.e. 100 μl of the suspension) were then injected into the caudal vein of C57BL/6 mice. 14 days after injection of the cells, the mice were sacrificed, the lungs were isolated and the metastases (spots positive for melanin) were counted under a dissection microscope.

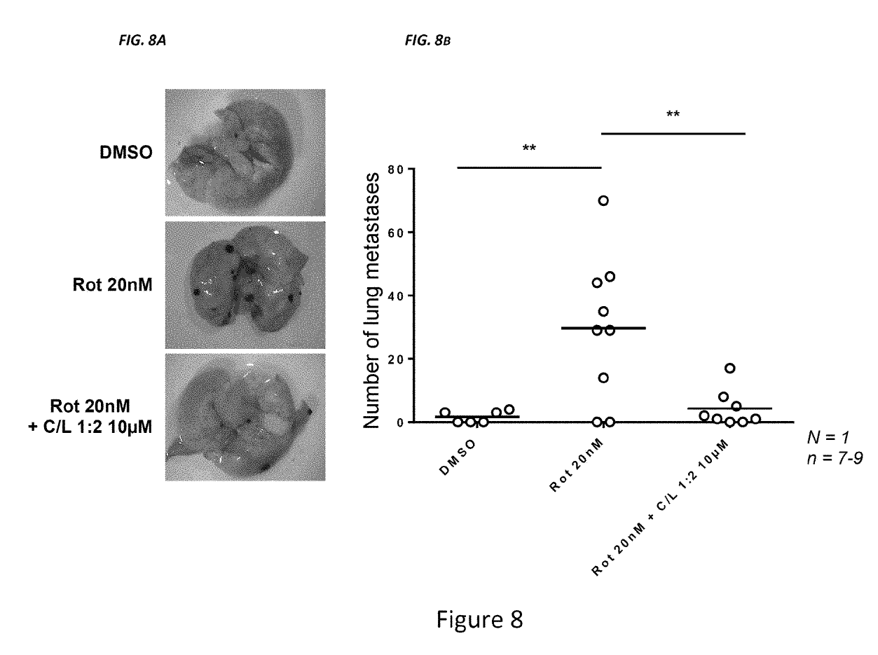

FIG. 8 presents representative photographs (A) and a graph (B) quantifying the formation of lung metastases 14 days after injection of 10⁶ B16F10 murine melanoma cells into the caudal vein of syngenic mice. The cells were pretreated with 0.5% DMSO (DMSO, negative control) or with the respiratory chain complex I inhibitor and mitochondrial superoxide inducer rotenone at 20 nM alone (Rot 20 nM, positive control) or in combination with 10 μM (+)-catechin/lysineHCl 1:2 (Rot 20 nM+C/L 1:2 10 μM). n=7-9, one-way ANOVA with Bonferroni test, ** p<0.01.

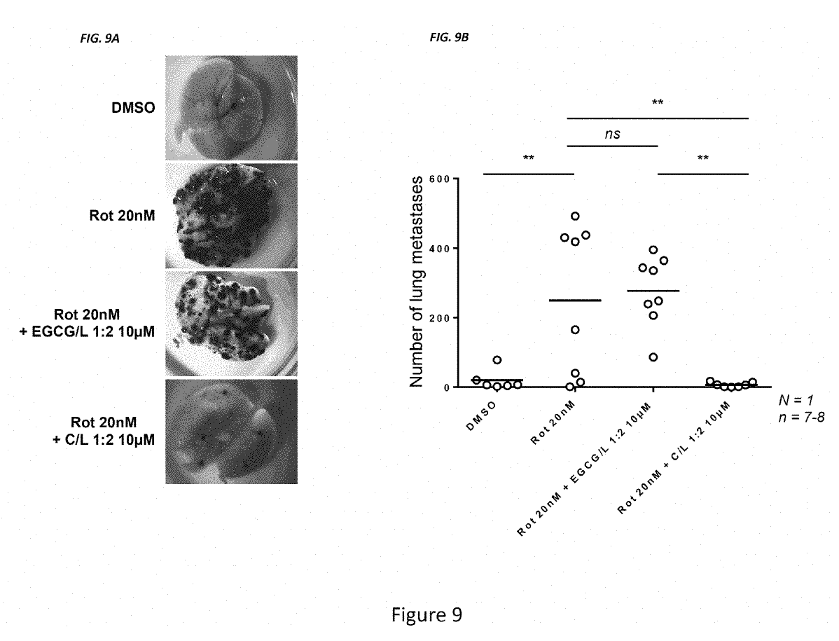

It is noted that the (+)-catechin/lysineHCl 1:2 inhibits the formation of metastases in an intravenous injection experimental model. FIG. 9 shows representative photographs (A) and the graph (B) quantifying the formation of lung metastases 14 days after injection of 10⁶ B16F10 murine melanoma cells into the caudal vein of syngenic mice. The cells were pretreated with 0.5% DMSO (DMSO, negative control) or with the respiratory chain complex I inhibitor and mitochondrial superoxide inducer rotenone at 20 nM alone (Rot 20 nM, positive control) or in combination with 10 μM (+)-catechin/lysineHCl 1:2 (Rot 20 nM+C/L 1:2 10 μM) or with epigallocatechin gallate/lysineHCl 1:2 (Rot 20 nM+EGCG/L 1:2 10 μM). n=7-8, one-way ANOVA with Bonferroni test, ns p>0.05, ** p<0.01.

It was observed that, contrary to the epigallocatechin gallate/lysineHCl 1:2, the (+)-catechin/lysineHCl 1:2 inhibits the formation of metastases in an intravenous injection experimental model.

### Example 8: H-Catechin/lysineHCl 1:2 but not Epigallocatechin Gallate Reduces the Formation of Lung Metastases in an Experimental Model

FIG. 10 shows representative photographs (A) and the graph (B) quantifying the formation of lung metastases 14 days after injection of 10⁶ B16F10 murine melanoma cells into the caudal vein of syngenic mice. The cells were pretreated with the respiratory chain complex I inhibitor and mitochondrial superoxide inducer rotenone at 10 nM in combination with epigallocatechin gallate (Rot 10 nM+EGCG 10 μM) or with 10 μM (+)-catechin/lysineHCl 1:2 (Rot 10 nM+C/L 1:2 10 μM). n=5-9, Student's t test, ns p>0.05, * p<0.05.

It was observed that the (+)-catechin/lysineHCl 1:2 inhibits the formation of metastases more effectively than the epigallocatechin gallate 1:2 in an intravenous injection experimental model.

Statistics:

In all of the experiments and examples 1 to 8, one-way ANOVA was applied followed by the appropriate multiple comparison tests (Dunnett for the in vitro tests, Bonferroni for the in vivo tests), and the Student's t test for the comparison of the two conditions presented in FIG. 10. A p-value <0.05 was considered to be statistically significant (*); ** indicates p<0.01; *** p<0.005. Ns (not significant) indicates that the p-value was >0.05. The columns of the in vitro graphs represent the mean of each group, the points of the in vivo graphs each represent one mouse and the horizontal bar represents the mean of each group. N indicates the number of times the experiment was reproduced independently, and n indicates the total number of replicates.

### Example 9: Anti-Metastatic Effect in a Model of Spontaneous Metastases

In order to study the metastatic process as a whole, a model of spontaneous metastases from a primary tumor is used (Porporato P E et al. Cell Rep 2014; 8:754-766). For example, in a model, 1 000 000 moderately metastatic murine melanoma cells (B16F10) or strongly metastatic murine melanoma cells (B16M4b, described in Porporato P E et al., Cell Rep 2014, 8:754-766), optionally carrying a luminescent or fluorescent reporter, are injected subcutaneously or intradermally into the flank of syngenic C57BL/6 mice. The anti-metastatic and/or antitumor activity of a compound of catechin type is evaluated by administering this compound to the animals, for example intraperitoneally or intravenously (injection) or orally (gavage, incorporation into food or into drinking water) and by comparing it to its carrier, according to a defined dosage regimen scheme (administration frequency and dose).

The growth of the primary tumor is carefully monitored by means of repeated measurements (for example every 2-3 days) of the tumor diameter by means of a caliper and/or of luminescence or fluorescence imaging in vivo if the expression of a reporter by the tumor cells enables this. When the mean tumor diameter reaches preferentially 10 mm (typically between days 10 and 14 for the abovementioned tumor lines), the primary tumor is excised with the double objective of keeping the mouse alive and of stimulating the development of the metastases (Porporato P E et al., Cell Rep 2014, 8:754-766; Gabri M R et al., Clin Cancer Res 2006; 12:7092-7098). The period of time between the surgeries on the first mouse and the last mouse to develop a primary tumor of required size is as short as possible (typically 5 days for the abovementioned tumor lines). The excision of a possible regrowth of the primary tumor is carried out as often as necessary, when this regrowth reaches the size of about 10 mm in diameter. In order to evaluate the metastatic dissemination, the mice are sacrificed in the various groups when one of the following events occurs: 1°) a first animal presents a state of suffering exceeding the ethical limits, 2°) a first animal dies without prior symptoms and exhibits numerous and/or bulky metastatic lesions, 3°) the monitoring of the luminescence or of the fluorescence in vivo indicates a considerable metastatic dissemination, if the cells express an appropriate reporter, 4°) a period of typically 30 to 40 days post-injection for the B16M4b mice or of typically 60 to 90 days for the B16F10 mice. In these melanoma models, the metastases essentially occur in the lungs and the lymph nodes (inguinal, axillary, mediastinal) and less frequently in the liver, on the peritoneum, and possibly the brain. For the quantification of the number of lung metastases, insufflation of the lungs by intratracheal injection of a saline solution is performed. The lung metastases (spots positive for the black melanin pigment) are counted using a dissection microscope 1. Alternatively, the ex vivo luminescence or fluorescence imaging of the organs removed and/or a histological analysis are carried out. In the case of a considerable variability in the size of the primary tumor, the results are standardized for each animal by means of the area under the curve of growth of the primary tumor until resection thereof.

It is observed that the catechin-type derivatives according to the invention have an anti-metastatic effect.

1-15. (canceled)

16. A method of preventing or treating cancer metastases in a subject, said method comprising, administering to said subject, a composition comprising at least one flavanol-type compound, wherein said at least one flavanol-type compound is (+)-catechin present in the form of a complex formed by the creation of coordinate bonds with at least one basic amino acid.

17. The method of claim 16, wherein said complex has a molar equivalence ratio between said (+)-catechin and said at least one basic amino acid of between 1:2 and 1:4.

18. The method of claim 16, wherein said at least one basic amino acid is lysine.

19. The method of claim 16, wherein said composition further comprises at least one acid.

20. The method of claim 19, wherein said acid is chosen from the group consisting of ascorbic acid, acetic acid, citric acid, hydrochloric acid, and mixtures thereof.

21. The method of claim 16, wherein said composition is characterized in that in a 0.01 molar solution at 25° C., it has a pH greater than or equal to 3, preferably of between 4 and 11, advantageously of between 4.5 and 7.5.

22. The method of claim 16, wherein said composition is in solid form.

23. The method of claim 22, wherein said composition is in water-soluble solid form.

24. The method of claim 23, wherein said composition is a powder or a table, a pessary or a suppository.

25. The method of claim 16, wherein said composition is administered orally.

26. The method of claim 16, wherein said composition further comprises one or more biocompatible excipients.

27. The method of claim 16, wherein said treatment is in combination with an anticancer treatment.

28. The method of claim 16, wherein said cancer is a liver, prostate, breast, uterine, testicular, bladder, kidney, lung, bronchial, bone, mouth, esophageal, stomach, pancreatic, colorectal or brain cancer.

29. The method of claim 16, wherein said cancer is a hepatocellular cancer.

30. The method of claim 16, wherein said cancer is a leukemia, a myeloma, a lymphoma or a melanoma.

31. The method of claim 16, wherein said composition is administered at a dosage of 250 mg to 750 mg/day.

32. The method of claim 16, wherein said composition treats circulating tumor cells.
